(19) United States
(12) Patent Application Publication
(10) Pub. No.: US 2006/0149121 A1
(43) Pub. Date: Jul. 6, 2006

(54) Instrument guide and method for use

(76) Inventors: James David Hughett SR., Liberty Twp., OH (US); Kenneth Lance Miller, Hamilton, OH (US); Salvatore Privitera, Mason, OH (US)

Correspondence Address: FROST BROWN TODD, LLC, 2200 PNC CENTER, 201 E. FIFTH STREET, CINCINNATI, OH, 45202, US

(21) Appl. No.: 11/028,901

(22) Filed: Jan. 3, 2005

(51) Int. Cl. A61F 2/00

(52) U.S. Cl. 600/37

(57) ABSTRACT

An instrument guide and method for use. An elongate flexible strip has a proximal end, a distal end, an a medial portion between the proximal and distal ends. A distal pocket is positioned adjacent the distal end of the elongate flexible strip. A medial pocket is positioned on the medial portion of the elongate flexible strip, the medial pocket being dimension to receive a surgical instrument. An anchor may be positioned adjacent the proximal end of the elongate flexible ribbon.

### BACKGROUND

The present invention relates to surgical instruments, with one embodiment relating to guides for surgical instruments. Surgery generally refers to the diagnosis or treatment of injury, deformity, or disease. Most surgical procedures involve the use of one or more surgical instruments. In most cases, the surgical instrument must be positioned on or near target tissue of the patient. Target tissue refers to the desired destination, whether final or intermediate, of a surgical instrument, and may or may not include the tissue that is the subject of surgery. A variety of factors may make the placement of the surgical instrument challenging. For example, the shape, position, or sensitivity of the target tissue or surrounding anatomy may complicate positioning of a surgical instrument. As a further example, the geometry of the surgical instrument may pose complications in positioning the instrument. In yet another example, the nature or timing of the procedure may complicate placement of the surgical procedure. Another example is the size of the surgical field. Still other examples of circumstances may complicate positioning of surgical instruments. Instrument guides are sometimes used to facilitate placement of surgical instruments. No one, however, has previously made or used an instrument guide in accordance with the present invention.

### BRIEF DESCRIPTION OF DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 illustrates an oblique view of an instrument guide;

FIG. 2 illustrates an oblique view of a bobbin for holding an instrument guide;

FIG. 3 illustrates an instrument guide with a positioning mechanism; and

FIG. 4 illustrates an instrument guide with a surgical instrument.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

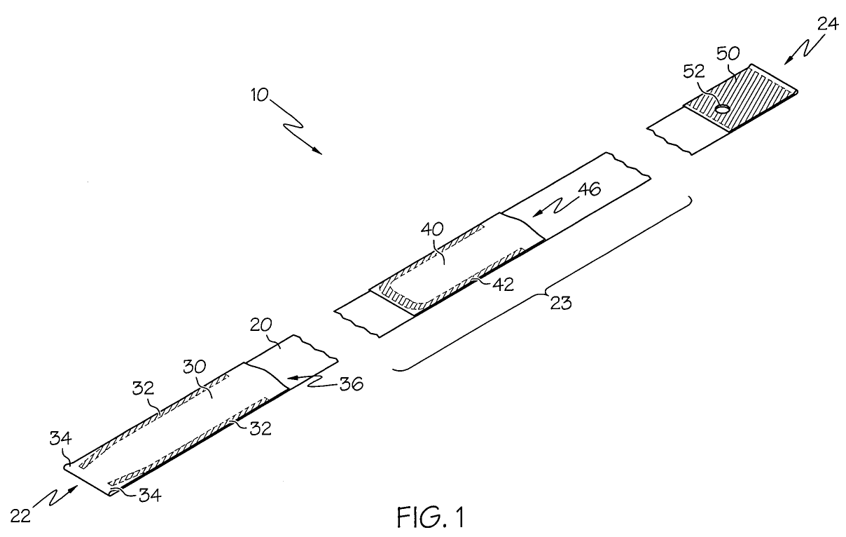

FIG. 1 illustrates an example of an instrument guide (10). The instrument guide (10) includes an elongate flexible strip (20) having a distal end (22), a proximal end (24), and a medial portion (23) between the proximal and distal ends. Optionally, the elongate flexible strip (20) is formed at least in part of an elastomer. One suitable material is a polyurethane, such as PELETHANE series 2363 by DOW PLASTICS, but other materials may also be used. In the present embodiment, the elongate flexible strip (20) is in the form of a ribbon; however, other geometries could also be employed. The dimensions of the elongate flexible strip (20) may vary substantially, and in this example the ratio of width to length is about 0.01 to about 0.03. In the present embodiment, the elongate flexible strip (20) is about 30 to about 40 inches long, about 0.4 to about 0.8 inches wide, and about 0.001 to about 0.005 inches thick. For example, one embodiment has a nominal length of about 36 inches, a nominal width of about 0.75 inches, and a nominal thickness of about 0.003 inches.

A first pocket (30) is positioned adjacent the distal end (22) of the elongate flexible strip (20). As shown here, the distal pocket (30) has an opening (36) on the proximal end of the pocket, and the distal end of the pocket is substantially closed. The distal pocket (30) is formed by folding the elongate flexible strip (20) onto itself and adhering the walls to form seals (32). Heat or RF sealing or use of an adhesive are two exemplary techniques to adhere the walls. As shown here, the seals (34) tapered toward the distal end of the pocket. Two tabs (34) are provided at the distal end (22). Optionally, at least a portion of the distal pocket (30) is translucent or transparent.

The present embodiment has a second pocket (40) positioned proximally to the distal pocket (30) on the elongate flexible strip (20). As shown here, the second pocket (40) is positioned on the medial portion (23) of the elongate flexible strip (20). In this embodiment, the medial pocket (40) has an opening (46) on the proximal end of the pocket, and the distal end of the pocket is completely closed. The medial pocket (40) is formed by placing a section of material over the elongate flexible strip (20), preferably but not necessarily the same material, and adhering the walls to form the seal (42). As shown in this example, the distance from the distal end (22) of the elongate flexible strip (10) to the distal end of the medial pocket (40) is between about 15 inches to about 30 inches. In one embodiment, the nominal distance is about 21 inches.

An anchor (50) is positioned adjacent the proximal end (24) of the elongate flexible strip (20). The anchor (50) of the present embodiment includes a hole or eyelet (52), but other attachment mechanisms may be employed, such as holes, slits, hooks, fasteners, etc. Further, the anchor may have more than one anchor position. As shown here, the anchor (50) is formed by folding the elongate flexible strip (20) onto itself and adhering the walls together, such as with a heat seal or adhesive, thus increasing the wall thickness of the anchor (50). One advantage of increased wall thickness is to reinforce the eyelet (52). As shown in this example, the distance from the distal end of the medial pocket (40) to the eyelet (52) is between about 12 inches to about 13 inches. One embodiment has a nominal distance of about 12.5 inches.

One exemplary use of the instrument guide (10) is to deploy a surgical instrument. A positioning mechanism is placed in the distal pocket (30), and then by using the positioning mechanism the distal pocket (30) is advanced adjacent target tissue. The distal pocket (30) is preferably dimensioned to receive the positioning mechanism. The type positioning mechanism may vary widely, depending, for example, on the geometry of the target tissue and the surrounding anatomy. For instance, the positioning mechanism could be a surgeon's finger, a dissector, forceps, an articulated probe, and the like. The positioning mechanism may then be removed from the distal pocket (30). The tabs (34) may facilitate the removal of the positioning mechanism by providing a surface that the surgeon may grasp, such as with their fingers or with forceps, and pull the distal pocket (30) from the positioning mechanism.

The surgical instrument is placed in the medial pocket (40). The surgical instrument will vary depending upon the treatment and procedure being conducted in the surgery. The medial pocket (40) is preferably dimensioned to receive the appropriate surgical instrument. The elongate flexible strip (10) is then advanced further, such as by pulling the distal end (22) until the surgical instrument is adjacent the target tissue. The surgical instrument is removed from the medial pocket (40), such as by continuing to advance the elongate flexible strip (10) while keeping the surgical instrument stationary. Thus, the surgical instrument is successfully positioned adjacent the target tissue. The remainder of the elongate flexible strip (10) may then be removed from the surgical field. Optionally, the elongate flexible strip (10) may remain in the surgical field, before or after the surgical instrument is deployed, and function as a sling to hold or position tissue. Preferably, the portions of the instrument guide (10) that contact tissue in the surgical field may be free of sharp or abrasive edges.

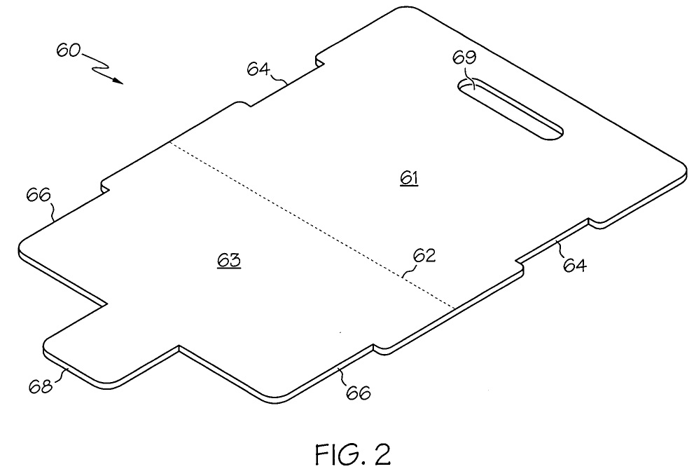

FIG. 2 illustrates an optional bobbin (60) for holding the instrument guide (10). The bobbin (60) comprises a sheet of material foldable along a crease (62) that separates a base portion (61) and a cover portion (63). The bobbin (60) has an opened position (as shown in the figure) and a closed position where the cover portion (63) is folded onto the base portion (63). In the closed position, the recesses (64, 66) align with one another. The tab (68) mates with the slot (69) to function as a fastener to selectively hold the bobbin (60) in the closed position. Naturally, alternative fastening mechanisms may also be used. As shown in this embodiment, the bobbin (60) is made from a single sheet of material, such as 0.01 inch LEXAN 8040. The sheet has a nominal width of about 3 inches and a nominal length of about 5 inches.

In the opened position, the instrument guide (10) is wrapped between the recesses (64) starting the with proximal end (24). Before the entirety of the elongate flexible strip (20) is wrapped, the cover portion (63) is closed and fastened, and the remainder of the instrument guide (10) is wrapped between the recesses (66). The medial pocket (40) may be wrapped between recesses (64) in the opened position; however, the medial pocket could also be wrapped between recesses (66) in the closed position. The completely wrapped instrument guide (10) and bobbin (60) may then be stored in a sterilized and sealed pouch with instructions. Once the pouch opened, the surgeon unwraps a portion instrument guide (10) without releasing the tab (68). Thus, the surgeon may work with the distal pocket (30) and a portion of the elongate flexible strip (20) while the remainder of the instrument guide (10) is contained in the closed bobbin (60) and out of the way during the surgery. Once the surgeon is ready to advance the surgical instrument into the surgical field, the bobbin (60) may be opened and the remainder of the instrument guide (10) unwrapped and used. The bobbin (60) may then be discarded.

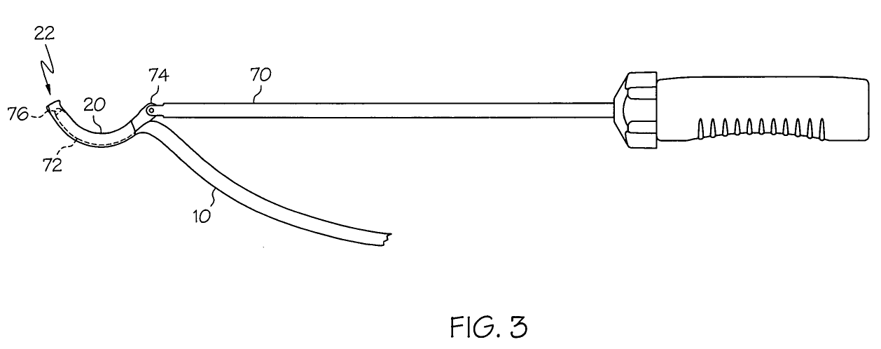

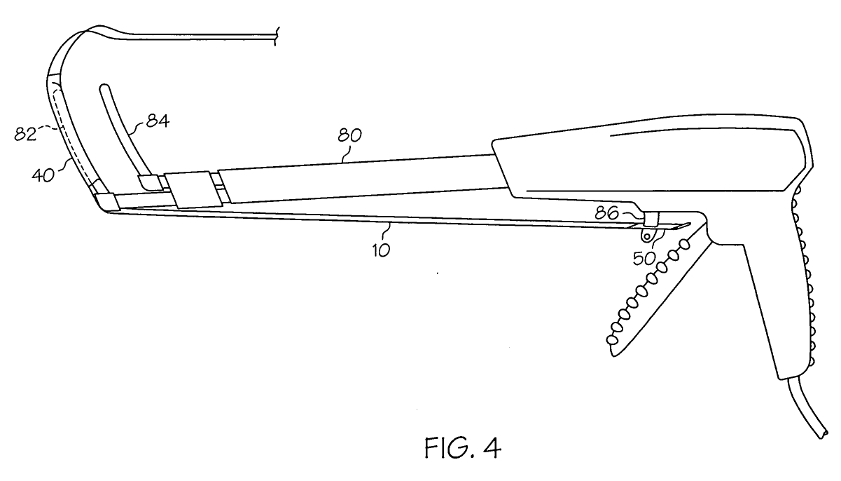

FIGS. 3 and 4 illustrates an example of a procedure to use the instrument guide (10) in conjunction with a treatment for atrial fibrillation. It should be appreciated that numerous other surgical procedures, anatomies, and surgical instruments may be used, and the present example is merely an illustration of one embodiment of the invention. The positioning mechanism (70) in this example, shown here as a dissector, is placed in the distal pocket (20). The positioning mechanism (70) has an articulated shaft and includes an arcuate segment (72) that pivots about the joint (74). The arcuate segment (72) has blunt and rounded distal end (76). A light source emits visible energy from the distal end (76) that facilitates, among other things, locating the distal end (76) during a procedure and differentiating tissue. In this example, the distal pocket (20) is translucent.

The following describes an exemplary procedure using the positioning mechanism (70) to separate the left or right pair of pulmonary veins adjacent the left atrium. The procedure may be performed during open or minimally invasive surgery. With the arcuate segment (72) in the distal pocket (20), the distal end (76) of the arcuate segment (72) is positioned adjacent the junction of one of the pulmonary veins (superior or inferior) and the left atrium. The distal end (76) is advanced around the posterior of the pair of pulmonary veins while simultaneously pivoting the arcuate segment (72). The distal end (76) continues to advance until it emerges beyond the other adjacent pulmonary vein (the inferior or superior, as the case may be). The advancement of the distal end (76) separates the pair of pulmonary veins from the pericardial reflections, thus creating a dissected path between the pulmonary veins and the pericardium. The dissected path may be widened by sweeping the arcuate segment (72) and further separating the tissue and widening the dissected path. The surgeon may grasp the distal end (22) of the instrument guide (10) and pull the distal pocket (20) from the arcuate segment (72). The arcuate segment (72) may then be backed out and removed from the surgical field.

As illustrated in FIG. 4, the surgical instrument (80) in this example is a surgical clamp having a distal jaw (82) and a proximal jaw (84). In this example, the surgical instrument (80) is used to ablate tissue with RF energy (one example is disclosed in U.S. Pat. No. 6,517,536). One of the jaws, in this example the distal jaw (82), is placed in the medial pocket (40) of the instrument guide (10). The anchor (50) attaches to surgical instrument (80) to prevent the distal jaw (82) from inadvertently liberating from the medial pocket (40). As shown in this example, the anchor (50) attaches to the clamp release lever (86) and the instrument guide (10) remains taut between the anchor (50) and medial pocket (40). By continuing to pull the distal end (22) of the instrument guide (10), the distal jaw (82) may be accurately positioned in the dissected path until the pulmonary veins are interposed between the distal and proximal jaws (82, 84). The anchor (50) may then be detached. By pulling instrument guide (10), the medial pocket (40) may be pulled from the distal jaw (82). The instrument guide (10) may be removed from the surgical field. Alternatively, the instrument guide (10) can remain in the surgical field to hold or lift the vessels like a sling. The distal and proximal jaws (82, 84) may then be positioned such that the tissue being treated is interposed between the jaws. In one example, the tissue being treated is the atrium wall adjacent the pulmonary veins. The jaws may then be closed and the tissue ablated.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A guide for a surgical instrument, comprising:
a) an elongate flexible strip having a proximal end and a distal end;
b) a first pocket positioned on the elongate flexible strip; and
c) a second pocket positioned on the elongate flexible strip proximally to the first pocket, the second pocket being dimension to receive a surgical instrument.

2. The guide of claim 1, wherein the strip is a ribbon.

3. The guide of claim 1, wherein at least a portion of the first pocket is translucent or transparent.

4. The guide of claim 1, wherein the first pocket comprises an opening at the proximal end of the pocket.

5. The guide of claim 1, wherein the first pocket is tapered toward the distal end.

6. The guide of claim 1, wherein the second pocket comprises an opening at the proximal end of the pocket.

7. The guide of claim 1, wherein the strip is an elastomer.

8. The guide of claim 1, wherein the elastomer is a polyurethane.

9. A guide for a surgical instrument, comprising:
a) an elongate flexible strip having a proximal end, a distal end, an a medial portion between the proximal and distal ends;
b) a distal pocket positioned adjacent the distal end of the elongate flexible strip; and
c) a medial pocket positioned on the medial portion of the elongate flexible strip, the medial pocket being dimension to receive a surgical instrument.

10. The guide of claim 9, wherein the strip is a ribbon.

11. The guide of claim 10, wherein the ratio of width to length of the ribbon is between about 0.01 to about 0.03.

12. The guide of claim 10, wherein the ribbon is a polyurethane.

13. The guide of claim 9, wherein the surgical instrument is a surgical clamp, and medial pocket is dimensioned to receive one jaw of the surgical clamp.

14. A method for deploying a surgical instrument using the guide of claim 9, comprising:
a) placing a positioning mechanism in the distal pocket;
b) using the positioning mechanism to advance the distal pocket adjacent target tissue;
c) removing positioning mechanism from the distal pocket;
d) placing the surgical instrument in the medial pocket;
e) pulling the elongate flexible strip until the surgical instrument is adjacent the target tissue; and
f) removing the surgical instrument from the medial pocket.

15. The method of claim 14, wherein the acts are performed sequentially.

16. The method of claim 14, wherein the surgical instrument is a surgical clamp and act of placing the surgical instrument comprises placing one jaw of the surgical clamp in the medial pocket.

17. The method of claim 16, wherein the surgical clamp is a tissue ablation tool.

18. The method of claim 17, wherein the target tissue is a pulmonary vein adjacent the heart.

19. The guide of claim 9, further comprising an anchor positioned on the elongate flexible strip proximally to the medial pocket.

20. The guide of claim 19, wherein the anchor comprises an eyelet.

21. The guide of claim 19, wherein the distance from the distal end of the medial pocket to the anchor is between about 10 inches to about 15 inches.

22. A bobbin for holding the elongate flexible strip of claim 9, the bobbin comprising a foldable sheet having a base portion and a cover portion, the foldable sheet having an opened position and a closed position, wherein a portion of the elongate flexible strip is wrapped around the base portion in the opened position and a portion of the elongate flexible strip is wrapped around the base portion and cover portion in the closed position.

23. The bobbin of claim 22, further comprising a fastener to selectively hold the foldable sheet in the closed position.

24. A guide for a surgical clamp, comprising:
a) an elongate flexible ribbon having a proximal end, a distal end, an a medial portion between the proximal and distal ends;
b) a distal pocket positioned adjacent the distal end of the elongate flexible ribbon, the distal pocket being opened on the proximal end of the pocket and closed at the distal end of the pocket, the distal pocket being translucent or transparent and being dimensioned to receive a positioning mechanism;
c) a medial pocket positioned on the medial portion of the elongate flexible ribbon, the medial pocket being opened on the proximal end of the pocket and closed at the distal end of the pocket, the medial pocket being dimension to receive one jaw of a surgical clamp; and
d) an anchor adjacent the proximal end of the elongate flexible ribbon.
